(19) United States
(12) Patent Application Publication
(10) Pub. No.: US 2021/0369136 A1
(43) Pub. Date: Dec. 2, 2021

(54) System & Method for Measurement of Respiratory Rate and Tidal Volume Through Feature Analysis of Breath Sounds to Detect Disease State

(71) Applicant: Florida Institute for Human & Machine Cognition, Inc., Pensacola, FL (US)

(72) Inventors: Jeffrey Brooks Phillips, Pensacola, FL (US); Anil Raj, Pensacola, FL (US); Bonnie Dorr, Pensacola, FL (US); Roger Carff, Pensacola, FL (US); Ian Perera, Pensacola, FL (US); Arash Golibagh Mahyari, Pensacola, FL (US); Michelle Sciarini, Pensacola, FL (US); Adrien Moucheboeuf, Pensacola, FL (US); Archna Bhatia, Pensacola, FL (US)

(73) Assignee: Florida Institute for Human & Machine Cognition, Inc., Pensacola, FL (US)

(21) Appl. No.: 17/320,748

(22) Filed: May 14, 2021

Related U.S. Application Data
(60) Provisional application No. 63/025,430, filed on May 15, 2020.

(51) Int. Cl. A61B 5/08; A61B 5/00; A61B 5/097

(52) U.S. Cl. CPC A61B 5/0803; A61B 5/0816; A61B 5/742; A61B 5/097; A61B 5/6898

(57) ABSTRACT

A system and method using a microphone to collect sound data produced by a potential patient's respiration and speech. The system preferably uses a microphone on a portable electronic device—such as a smart phone. The analysis of the collected data is preferably performed locally—such as by a software application running on the smartphone. The software is used to analyze the data and therefore determine and track useful parameters such as respiration rate and respiratory tidal volume. The software also analyzes phonation patterns. Using the parameters, the inventive system can detect the onset of respiratory distress.

### CROSS-REFERENCES TO RELATED APPLICATIONS

This non-provisional patent application claims the benefit of an earlier-filed provisional application. The provisional application was assigned U.S. Ser. No. 63/025,430. It listed the same inventors.

### STATEMENT REGARDING FEDERALLY SPONSORED RESEARCH OR DEVELOPMENT

Not applicable.

### MICROFICHE APPENDIX

Not Applicable

### BACKGROUND OF THE INVENTION

### 1. Field of the Invention

The present invention pertains to the field of human health. More specifically, the invention comprises a system and method for measuring human respiratory rate and tidal volume using analysis of sounds produced by respiration and speech, as well as possibly other parameters.

### 2. Description of the Related Art

Respiratory distress is an early indicator of many human diseases. At the present time there is great interest in the early detection and triaging of the infectious disease known as COVID-19. Over the past three months COVID-19 has resulted in approximately 4 million infections and three hundred thousand deaths and has brought the world economy to a halt, resulting in unemployment unparalleled since the Great Depression of the 1930s. Due to its extremely infectious nature and ability to be transferred by infected individuals who are asymptomatic, new and innovative methods to identify the early signs of infection and to monitor infected patients while in their homes are needed. Currently available early detection technologies—such as pulse oximetry—are expensive and difficult to scale for wide and rapid distribution.

Respiratory distress is now known to be an early indicator of COVID-19 infection. In many cases, mild respiratory distress can exist for a period before the patient perceives it. The detection of this early-stage distress would allow healthcare providers to better triage and treat these patients.

Early respiratory distress can be detected via the measurement of respiration rate, breathing sounds, and speech patterns. Experienced clinicians—who are familiar with the progression of respiratory distress in various diseases—can often subjectively detect the alteration of breathing sounds and alteration of speech. Respiration rate is of course an objective parameter that can be measured.

The inventors believe it is possible to automatically detect COVID-19 symptomology and severity through the collection of sound data and the subsequent analysis of respiration rate, the detection of abnormal breathing sounds, and the detection of abnormal speech. An increasing respiration rate is one of the earliest signs of COVID-19 patient deterioration. Monitoring the respiration rate provides the ability to predict significant problems 24 hours in advance, giving caregivers additional time for treatment and stabilization. Frequency and duration-based features can also help distinguish abnormal breath sounds from normal breath sounds.

Spectral analysis has been shown to be useful for classifying lung sounds as well as speech associated with conditions such as pneumonia. Spectral density and amplitude can be indicative of the state of the lungs and dimensions of the airways. Similarly, acoustic and durational features of speech—such as phonation times, greater jitter, reduced harmonic-to-noise ratio, and reduced phonation range, have also been associated with conditions such as chronic cough.

The inventors believe that the present inventive system has the potential to detect early respiratory distress—in some cases before the patient is even aware of its existence. Thus, the present invention allows for early detection of possible COVID-19 infections. This early detection capability allows the referral of the patient to disease-specific testing.

The inventive system and method will provide a valid, reliable COVID-19 diagnostic and patient monitoring tool that can be easily distributed to any patient with a smartphone. This will allow individuals who have been exposed to identify their symptoms as early as possible so that they can be isolated to prevent the spread of the contagion. The application can then be used to provide continuous monitoring of infected individuals to identify early signs of deterioration that require critical medical attention.

### BRIEF SUMMARY OF THE INVENTION

The present inventive system and method uses a microphone to collect sound data produced by a potential patient's respiration and speech. The system preferably uses a microphone on a portable electronic device—such as a smart phone. The analysis of the collected data is preferably performed locally—such as by a software application running on the smartphone. The software is used to analyze the data and therefore determine and track useful parameters such as respiration rate and respiratory tidal volume. The software detects and monitors lung abnormalities based on feature analysis of sounds associated with three practical mechanisms that are complementarily informative of the status of the lungs. These are: normal breathing, breathing during speech production, and produced speech. Using the parameters, the inventive system can detect the onset of respiratory distress. The invention preferably includes alert features so that the patient or another person is alerted when respiratory distress is detected.

### BRIEF DESCRIPTION OF THE SEVERAL VIEWS OF THE DRAWINGS

FIG. 1 is a perspective view, showing an embodiment of the present inventive method implemented on a smartphone.

FIG. 2 is a block diagram, showing internal components present in the smartphone depicted in FIG. 1.

FIG. 3 is a plot of preliminary testing in which the inventive algorithm was applied to twelve different samples of breathing sound data.

### DETAILED DESCRIPTION OF THE INVENTION

The inventors are presently developing a breath and speech processing system that will significantly improve the early diagnosis of COVID-19 and improve the monitoring of COVID-19 patients using a simple and inexpensive smartphone application. The invention is potentially applicable to many other human conditions and the scope of the present invention should not be viewed as limited to COVID-19. However, because COVID-19 is presently a substantial public health concern, the examples provided will be directed to that disease.

Using the inventive system and method, COVID-19 symptomology and severity will be detected through the analysis of respiration rate (RR), (abnormal) breathing sounds, and speech over a smartphone application and unobtrusive microphone. RR is one of the earliest signs of patient deterioration and possesses the ability to predict significant problems 24 hours in advance, giving caregivers additional time for treatment and stabilization. Frequency and duration-based features can help identify abnormal breath sounds from normal breath sounds.

Spectral analysis has been shown to be useful for classifying lung sounds as well as speech associated with conditions such as pneumonia. Spectral density and amplitude can be indicative of the state of the lungs and dimensions of the airways. Similarly, acoustic and durational features, such as phonation times, greater jitter, reduced harmonic-to-noise ratio, and reduced phonation range, in speech have also been associated with conditions such as chronic cough.

Preliminary data have already shown that the inventive system can identify lung sounds and breathing disruptions found in COVID-19 affected individuals. The project goal is the fast development of a smartphone application that analyzes acoustic, prosodic and durational features of breath and speech to provide a sensitive diagnostic tool to identify the early signs of COVID-19 infection. The application will also be used to provide continuous monitoring of COVID-19 positive patients following diagnosis for early detection of patient deterioration.

The inventors propose to use samples of speech produced by individuals to triage patients for definitive COVID-19 testing (i.e., blood or nasopharyngeal swab). Because the respiratory system generates the required air flow for speech production, changes in lung capacity and function can qualitatively impact speech production by an individual. In fact, health care professionals can estimate lung function with a median error of 10.6% based on listening to recorded samples of speech (Tayler 2015), suggesting that speech can indeed be indicative of lung function.

Hypoxia has also been identified as a feature of COVID-19, sometimes occurring without acute respiratory distress. Sustained hypoxia from hemoglobin oxygen saturation (SpO2) percentages below 94% can disturb brainwave patterns (Rice et al, 2019) and interfere with cognitive performance, including speech cadence, disfluencies, word selection, etc. Similarly, breathing samples will be obtained from participants for identifying any abnormalities in RR and breathing sounds that can be indicative of affected lung function in COVID-19 patients.

A software system that extracts and models the underlying features of breath and speech could process an individual's breath and speech to screen individuals for definitive molecular and/or serological tests for COVID-19 while these tests are in short supply. In addition, by deploying the software system as a smartphone application (App), it could provide cost-effective sampling of multiple data points over time to identify breath and speech feature trends that correlate with worsening (or improving) lung function, rather than the intermittent snapshots provided by current testing methods. We expect that computational modeling of changes in breath and speech based on these features will perform better on this COVID-19 detection task than healthcare providers or contact tracers could when observing individuals speak and breathe or when asking if they noticed changes/problems with speech and breathing.

Because speech is produced in natural settings and breathing is a regular activity performed continuously, a speech- and breathing-based test can be used anywhere and as frequently as deemed useful without consumables' costs associated with supplying or conducting the test. In fact, one of the limitations of molecular and serological tests is that people infected with SARS-CoV-2 who are asymptomatic may never get tested. The feasibility and easily widespread availability of a speech- and breathing-based test App, would make it useful for population surveillance for the developing COVID-19 in asymptomatic individuals (whether they are asymptomatic due to being in early phases of the disease or due to being generally healthy enough to fight the infection). As any molecular and/or serological tests may also give some false positive or false negative results, repeated sampling using this additional speech- and breathing-based test App, could provide further support to increase confidence in or refute a lab test result.

Thus, the application will be developed and refined using breath and speech sound recordings of human participants with COVID-19 and healthy individuals. Crossover experimental trials will also be conducted on healthy human participants while breathing mixed gasses that induce COVID-19 relevant physiological conditions (i.e., hypercapnia and hypoxia). Participants will be asked to perform speech related tasks (read passages and count loud) and breathe under both normal and experimental conditions. Divergent features will be identified between normal and diseased or stressed samples of breathing and speech. RR and breathing dynamics will be calculated by identifying divergent features that can distinguish inspiration from expiration in both normal and stressed conditions. RR and breathing dynamics will be validated using gold standard laboratory measures of RR and volume.

The inventors intend to deliver a detailed technical report describing all experiments and the sensitivity and specificity of the application for correctly identifying disease and stressed states and comparisons of breath-based measures (consisting of RR and respiration dynamics), and a speech-based measure (consisting of a subset of identified acoustic, prosodic and durational features) with gold standard laboratory instruments. The inventors also plan to deliver the smartphone application with a detailed instruction manual and recommendation on microphones to enhance the application in specific situations (i.e., nasal cannula, oxygen mask, or free breathing/speech).

The proposed study will test the hypotheses that (i) features in normal breathing, breathing during speech production and produced speech can identify COVID-19 in affected individuals, and (ii) they can also be used to measure severity of the condition in the affected individuals, and act as non-invasive measures of lung and respiratory function at a specific point in time. Additionally, the study will test the hypothesis that models of features in speech and breathing can also be used to identify the condition even before individuals manifest any physiological symptoms (fever, dyspnea, etc.). Prior clinical studies for certain neurological conditions (such as Amyotrophic Lateral Sclerosis/ALS) have demonstrated that speech features change well before individuals begin to show the symptoms that lead to diagnosis of the condition (Yorkston et al 1993). Bhatia et al (2017a, 2017b), while studying speech produced by ALS affected individuals, have shown that features in speech can be indicative of lung functioning (e.g., Observed Forced Vital Capacity) of ALS affected individuals, which helps detecting presence of ALS in individuals as well as providing a measure of its severity. Similarly, RR can detect problems hours before they are detectable through other vital signs.

A stepwise series of protocols will be used to collect recorded speech and breath samples from human research participants. For each study, the inventors will sample continuous speech from individuals when reading a passage out loud (read speech task) and loud speech when counting loudly (loud speech task) to study the impact of the condition on a range of speech mechanisms and features (dependent variables). Similarly, normal breathing samples and other parameters will be collected from individuals in each study. Additionally, clinical data will be collected to provide ground-truth physiologic context (independent variables). Analysis of the data will test the hypotheses and find correlations between the dependent and independent variables. The technique will include extraction of acoustic, prosodic and durational features in the speech samples, as well as respiration rate, pauses, disfluencies, etc., and their correlations with the independent variables to build models for COVID-19 screening (study 1) or detection of underlying mechanisms (study 2) using statistics and machine learning (ML).

Additionally, ML and time series models will be built to detect and predict an individual's prognosis for progression of their COVID-19. The outcome of the proposed studies will be models for detecting COVID-19 or related underlying mechanisms and severity in individuals to be used as a speech- and breathing-based COVID-19 diagnostic and monitoring app for a smartphone.

The entire inventive system is preferably made to be downloaded to a smartphone as an “app.” This approach obviates the need for mass manufacturing of electronics or other hardware and facilitates rapid distribution at the end of the period of performance.

The smartphone app will analyze features in breath and speech to identify early signs of COVID-19 such as increased RR, disrupted/impacted speech, and abnormal lung and bronchial sounds. The inventors plan to develop and refine the application by identifying characterizing features in breathing and speech for healthy and COVID-19 infected individuals as well as the divergence in features between healthy and COVID-19 infected individuals. The inventors will also model the divergence in features associated with relevant physiological states like hypoxia, hypercapnia, and tachypnoe. The application's sensitivity and specificity to identify the relevant disease and physiological states will be analyzed and validated by laboratory gold standards.

Respiration rate (“RR”) is the singular vital sign that provides the earliest warning of patient deterioration. RR can detect problems hours before they are detectable through other vital signs. In fact, RR has been shown to reliably predict emergency room visits 24 hours in advance. When used properly RR can significantly expand the time caregivers have to respond to acute threats and facilitate positive patient outcomes. As the earliest sign of patient distress, it has long been recommended that RR be measured accurately to provide the most robust approach to patient monitoring possible.

Unfortunately, RR is underutilized in patient monitoring due to reliability problems related to how it is measured. As standard practice, RR is measured by a caregiver counting the number of breaths a patient takes per minute. Rather than measuring RR for a full minute, caregivers are often limited to only fifteen to thirty seconds due to workload restrictions and distractions, negatively impacting the validity of RR. Caregivers are often unaware of the importance of RR, so they simply write in a value. Reliability issues have led to a general dismissal of RR as a tool for patient monitoring.

The development of a simple, reliable, and field expedient measure of RR would be a significant advancement in patient monitoring and a powerful diagnostic and early monitoring tool for diseases like COVID-19. Features can be used to identify inhalation and exhalation frequency, intensity, and duration (based on sound). This information will be utilized to provide a moving average of breath rate, breath duration, and inhalation and exhalation dynamics. Trends will be tracked over time which will significantly improve our techniques sensitivity to patient status.

The inventors have extracted frequency, time-frequency features, and Mel frequency (a transformation of frequency to the perception-based Mel scale) to represent the patient's breathing patterns. Feature selection algorithms and used to identify significant frequency and Mel frequency components. The identified significant features are used to train a classifier algorithm to distinguish and recognize COVID-19, hypercapnia, and other breathing patterns. In addition, linear regression is used to predict the percentage of carbon dioxide level, the respiration rate, and the today volume from the frequency and Mel components extracted from the breathing sounds.

Further, lung volumes and breathing patterns for quiet respiration have been found to be different from when speech is produced indicating that production of speech may involve lungs in ways that may be different from quiet respiration. Hence breathing patterns during speech production as well as features of produced speech itself can be expected to complement information obtained through normal respiration without speech. The complementary features of sounds produced through each of the three mechanisms (normal breathing, breathing during speech production and produced speech) can be combined to develop a more robust measure of lung abnormalities associated with COVID-19 infections which can result in compromised lung capacity, with 14% of the affected cases being severe enough to lead to Acute Respiratory Distress Syndrome, and even death (WHO 2020, WebMD 2020).

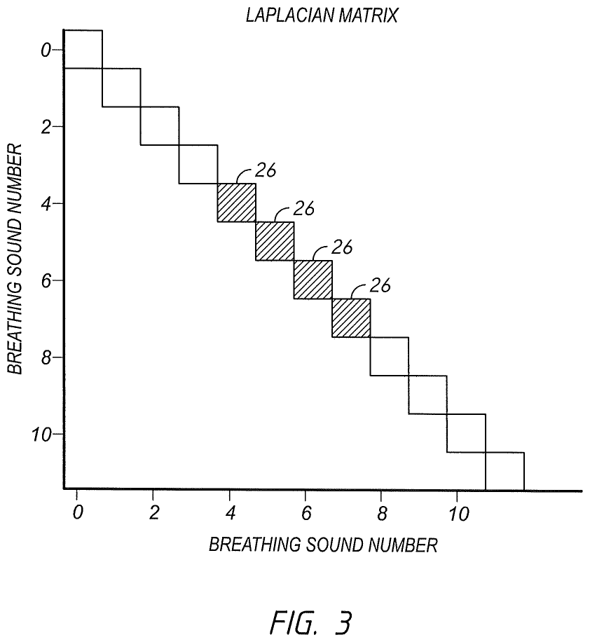

The inventors have implemented a prototype of the proposed inventive algorithm and evaluated it on a set of breathing sounds: normal (Vesicular Breath Sound), Rhonchi, and COVID-19. There are four 10-second segments of breathing sound for each condition. The first 4 audio signals are normal breathing sounds, signals 5 to 8 are COVID-19 sounds, and signals 9 to 12 are Rhonchi sounds. FIG. 3 shows the preliminary results of the algorithm development. As shown in the figure, the algorithm can clearly identify 3 clusters of Vesicular Breath Sound, Rhonchi, and COVID-19. Four middle entities 26 in the LaPlacian matrix are associated with COVID-19.

The inventors have also developed a prototype for speech features-based algorithms for detection and severity measurement of a condition, ALS, which also impacts the lungs (besides other physiological changes). Bhatia et al (2017a, 2017b) extracted acoustic, prosodic and durational features from the speech produced by ALS patients and by healthy individuals to identify most informative features for characterization of ALS through Pearson's Correlation Coefficient. These features were then used in the algorithms for detection and severity measurement of ALS. The developed models for ALS will be adapted and refined for COVID-19 detection and severity.

For the proposed work, two kinds of studies will be conducted: (i) a study to model underlying mechanisms (hypercapnia, hypoxia) to cause symptoms of COVID-19 infections, (ii) a study to identify speech and breathing feature signatures of COVID-19 infections in patients.

For the first study, the speech and breath samples will be collected from healthy individuals in a number of experimental (stressed) conditions for both hypercapnia and hypoxia exposure. For example, for hypercapnia, a mixed gas system will deliver gas mixtures of 1.0% CO2, 2.5% CO2, 4.0% CO2, and 5.5% CO2; and two non-stressed baselines will be used: (1) normal CO2 level without a mask (normal air) and (2) normal CO2 level with an aviation mask. The extracted features and/or divergence scores computed from extracted features in experimental and baseline conditions will be used for detection of physiological stress induced by these conditions.

For the second study, the speech and breath samples will be collected from known COVID-19 affected hospitalized patients and age matched known healthy individuals (COVID-19 positive and COVID-19 negative individuals, respectively). The extracted features and/or divergence scores computed from extracted features in samples from COVID-19 positive and COVID-19 negative individuals will be used to identify speech and breathing feature signatures of COVID-19 and its severity.

Additionally, the inventors plan to track a subset of the participants in the second study longitudinally to identify changes in speech and breath patterns as the infection evolves. We expect to cover cases where: (i) an individual has the condition at the beginning of the study and starts the recovery process during the study, (ii) an individual is not symptomatic at the beginning of the study but progresses during the study, and (iii) an individual does not have the condition through the duration of the study. The longitudinal time series data will be used to build models that track and predict changes in the condition (i.e., further development of clinical symptoms or their resolution).

The study participants will be suitably selected. Any individuals who are not able to speak or follow directions will be excluded from the study since the development of the proposed diagnostic test requires speech samples based on the protocol from the participants in addition to the breath samples. While inclusion criteria will not restrict the participants to any specific demographic groups based on age, gender, race or languages spoken etc., this information will be collected and used to help build more informed models, and in future work also to build more specific models for each different population. Similarly, information such as history of respiratory problems, allergies, smoking/non-smoking, voice disorders, formal training in singing and speaking, height, and weight, will also be collected.

The inventors plan to collect data from 60 healthy individuals for the first study, and 100 individuals consisting of evenly split COVID-19 positive and COVID-19 negative individuals for the second study. The inventors plan to follow 60 of the participants from the second study longitudinally for eight months. To account for attrition, besides the determination of COVID-19 positive and negative will be made at the time of recruitment from each individual's clinical test result, and information about any changes to that determination during the study will be recorded. The inventors anticipate that some test-negative individuals may develop the disease or test-positive individuals may recover from it during the study (depending on their stage and severity). The information about changes in condition will provide useful time series data for the longitudinal study.

In addition to a subset of participants from the second study mentioned above, the longitudinal study will consist of a subset of participants from the first study, however other participants who did not participate in the first study may also be recruited to account for attrition.

### Hypercapnia and Hypdxia Experiments

Participants will read passages aloud for 30 minutes in a non-stressed condition. The reading content will consist of 10 repetitions of the same passages. In a separate session, participants will don a standard flight mask, and in addition to the non-stressed condition with the mask, they will breathe four different gas mixtures for a period of 15 minutes per mixture (stressed condition). A mixed gas system will deliver gas mixtures of 1.0% CO2, 2.5% CO2, 4.0% CO2, and 5.5% CO2. In a separate session participants will be seated inside a normobaric altitude chamber and breathe O2 concentrations of 14.3%, 13.20%, 12.3%, and 11.4%. Breath and reading samples will be recorded throughout baseline and both physiological stress exposures.

### Analysis

Algorithm accuracy will be evaluated in three steps. First, sample wide feature reduction will be accomplished by examination of correlations between the aforementioned 4,000 vocal/breath features and arterial CO2 and O2 state followed by a Principal Components Analysis. Second, divergence scores will be calculated within each operator using the values for the reduced set of acoustic and duration-related features in an operator's vocalizations and breath in non-stressed vs stressed conditions. A classification of the most robust features and divergence scores will be performed using machine learning algorithms, such as Support Vector Machine and logistic regression, to detect hypercapnia. In order to build the machine learning models, 80% of the data will be used for training, and 20% will be held out as test data. Ten-fold cross validation will be used to train the models on the 80% training data. The trained models will then be evaluated on the previously held out test data. The train-test split of the data will be performed in two ways (between the subjects and within the subjects) to conduct two different experiments.

For the first experiment, the split will be between the subjects. The inventors plan to hold out data from 20% of the randomly selected subjects (N=10) for validation and the data from the rest of the 80% subjects (N=40) will be used for training. For this scenario, the inventors will train and test 10 times corresponding to each iteration of reading passages and will average out the accuracies. A good accuracy in this experiment will indicate robustness of our algorithm and generalizability to the general population.

For the second experiment, the split will be within speakers, such that 20% of the data from each speaker (2 repetitions of the reading passages) will be held out as test and the remaining 80% (8 remaining repetitions) will be used for training. This experiment will help the inventors determine how well the algorithm learned the patterns associated with different experimental conditions (i.e., levels of CO2). This approach tests an individual against their own established baseline, so that features associated with pre-existing conditions can be controlled for in patients with significant comorbidities.

Measurements of RR and breathing dynamics will be validated through direct comparisons to laboratory gold standards through Bland-Altman analyses. ROC Curves will be produced to determine the algorithm's sensitivity and specificity in identification of COVID-19 related physiological states.

A study will be conducted to identify speech and breathing feature signatures of COVID-19 by collecting data from known COVID-19 affected hospitalized patients and age matched known healthy individuals (COVID-19 positive and COVID-19 negative individuals, respectively). Acoustic, prosodic and durational features in speech and breathing will be used for COVID detection and severity.

The inventors will track a subset of the participants longitudinally to identify changes in speech patterns as the infection evolves. We expect to cover cases where: (i) an individual has the condition at the beginning of the study and starts the recovery process during the study, (ii) an individual is not symptomatic at the beginning of the study but progresses during the study, and (iii) an individual does not have the condition through the duration of the study. The longitudinal time series data will be used to build models that track and predict changes in the condition (i.e., further development of clinical symptoms or their resolution).

The inventors anticipate that the inventive COVID-19 Smartphone App will provide reliable and valid identification of the earliest signs of COVID-19 in an inexpensive, unobtrusive, and easily scalable field deployable package. It will allow for automated screening with easily interpretable results so that COVID-19 infected individuals can be identified early and isolated to prevent additional disease transmission. Once infected the smartphone app can be used to monitor an individual's speech, lung sounds, RR, and respiratory dynamics to identify patient deterioration as early as possible so that urgent care can be provided before patients become critical.

### Exemplary Smartphone Application

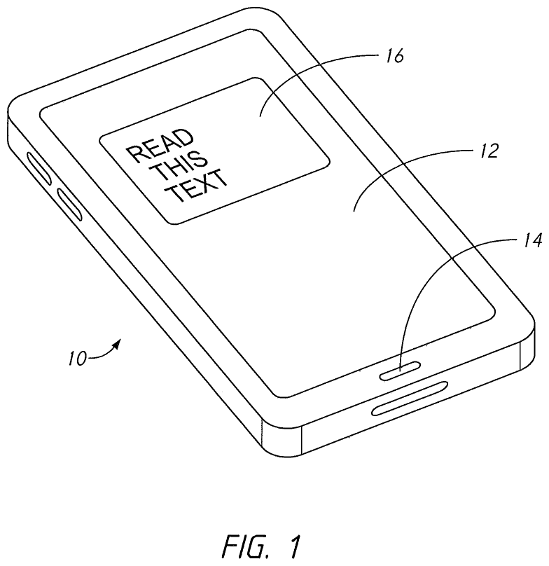

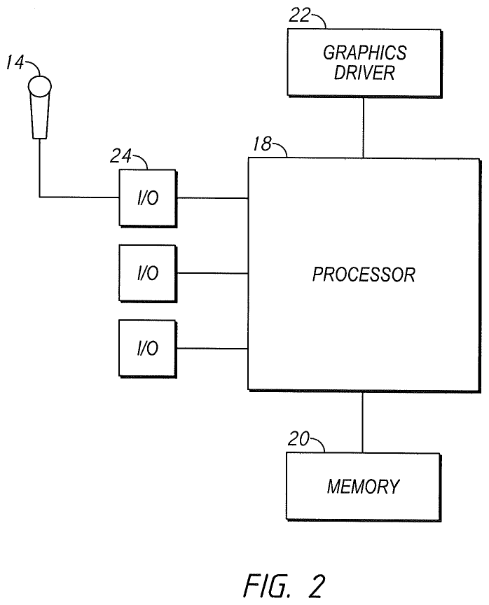

The inventive software is preferably implemented as an application running on a portable device such as a smartphone. FIG. 1 depicts a prior art smartphone 10 having a touchscreen display 12 and a microphone 14. FIG. 2 provides a greatly simplified depiction of the internal components of the smartphone. Processor 18 runs software—such as the inventive application—that is stored in associated memory 20. Graphics driver 22 causes the display of a graphical user interface on the smartphone's display. Input/output ports 24 provide inputs to the processor. One of these inputs is microphone 14.

Returning to FIG. 1, the smartphone causes a suitable graphical user interface 16 to be displayed on touchscreen display 12. One of the diagnostic approaches used in the present invention requires the user to read a piece of text while the microphone 14 records sound data. The text can be presented in a way that regulates the rate at which the user reads the text aloud. As an example, the text can be scrolled from bottom to top within the text window shown in FIG. 1.

A person must exhale lung volume while reading words aloud. This requires the person to periodically inhale. This inhalation is generally made very rapidly—as the person must pause speaking while inhaling. The result is a sharp inhalation that can be of significant diagnostic value. In addition, the rate and timing of the inhalations can be significant.

In the preferred implementation the user is prompted to record a series of “baseline” readings while in a known, healthy state. The sound data is recorded in memory. Optionally, only the parameters derived from the sound data may be stored in memory. As an example, the average interval between inhalations (while speaking a given piece of text) can be recorded (or a derived value for the respiration rate). This data becomes a “baseline.” The user is later asked to read aloud the same piece of text and a new current value for the average interval between inhalations is determined. If the average interval is decreasing over time, this indicates the onset of shortness of breath. If the respiration rate is used, then an increase in the respiration rate is used to indicate shortness of breath.

While it is desirable to have a baseline test performed when the user is known to be healthy, a baseline created at the onset of a disease process is still helpful. The user may perform a baseline test when already short of breath. That baseline is still helpful in allowing the software to determine whether the condition is growing worse or improving.

Inhalations during speech are particularly easy to detect, even with a small microphone such as found on a smartphone. The sharp inhale of breath is a distinctive sound and software can positively identify this sound across a broad range of users.

Of course, the smartphone application can perform more complex analyses than determining the average interval between inhalations. Preferably the inventive software records sound and analyzes that sound during inhalations and during speech production. In addition, the inventive software preferably analyzes the speech itself to detect changes in speech patterns that are associated with shortness of breath. The software extracts frequency, time-frequency features, and Mel frequency to represent the breathing patterns. Feature selection algorithms are used to identify significant frequency and Mel components. The identified significant features are used to train a classifier to distinguish and recognize COVID-19, hypercapnia, and other breathing patterns. In addition, linear regression is used to predict the carbon dioxide level, the respiration rate, and the tidal volume from the frequency and Mel components (extracted from the breathing sound).

The preceding description contains significant detail regarding the novel aspects of the present invention. It should not be construed, however, as limiting the scope of the invention but rather as providing illustrations of the preferred embodiments of the invention. Thus, the scope of the invention should be fixed by the claims ultimately drafted, rather than by the examples given.

Having described our invention, we claim:

1. A method for monitoring human respiratory performance of a user, comprising:
(a) providing a smartphone having a processor, a memory associated with said processor, a microphone configured to feed sound data to said processor, and a display;
(b) providing a software application stored within said memory and configured to run on said processor;
(c) using said software application to display a passage of text to said user on said display;
(d) said software application receiving sound data from said microphone as said user reads aloud said passage of text; and
(e) said software application using said sound data to determine a current state of said human respiratory performance for said user.

2. The method for monitoring human respiratory performance of a user as recited in claim 1 comprising:
(a) storing said current state of said human respiratory performance over time; and
(b) said software application determining a change in said human respiratory performance.

3. The method for monitoring human respiratory performance of a user as recited in claim 1 comprising said software application monitoring for inhalations while said user reads aloud said passage of text.

4. The method for monitoring human respiratory performance of a user as recited in claim 3 comprising said software application using said inhalations to determine a respiration rate for said user.

5. The method for monitoring human respiratory performance of a user as recited in claim 2 comprising said software application monitoring for inhalations while said user reads aloud said passage of text.

6. The method for monitoring human respiratory performance of a user as recited in claim 5 comprising said software application using said inhalations to determine a respiration rate for said user.

7. The method for monitoring human respiratory performance of a user as recited in claim 6, comprising said software application determining a change in said human respiratory performance by determining a change in said respiration rate over time.

8. The method for monitoring human respiratory performance of a user as recited in claim 1 wherein said software employs frequency analysis.

9. A method for monitoring human respiratory performance of a user, comprising:
(a) providing a smartphone having a processor, a memory associated with said processor, a microphone configured to feed sound data to said processor, and a display;
(b) providing a software application stored within said memory and configured to run on said processor;
(c) using said software application to display a passage of text to said user on said display;
(d) said software application receiving sound data from said microphone as said user reads aloud said passage of text; and
(e) said software application using said sound data to determine a current state of breath rate, breath duration, and inhalation and exhalation dynamics for said user.

10. The method for monitoring human respiratory performance of a user as recited in claim 9 comprising:
(a) storing said current state of said human respiratory performance over time; and
(b) said software application determining a change in said human respiratory performance.

11. The method for monitoring human respiratory performance of a user as recited in claim 9 comprising said software application monitoring for inhalations while said user reads aloud said passage of text.

12. The method for monitoring human respiratory performance of a user as recited in claim 11 comprising said software application using said inhalations to determine a respiration rate for said user.

13. The method for monitoring human respiratory performance of a user as recited in claim 10 comprising said software application monitoring for inhalations while said user reads aloud said passage of text.

14. The method for monitoring human respiratory performance of a user as recited in claim 13 comprising said software application using said inhalations to determine a respiration rate for said user.

15. The method for monitoring human respiratory performance of a user as recited in claim 14, comprising said software application determining a change in said human respiratory performance by determining a change in said respiration rate over time.

16. The method for monitoring human respiratory performance of a user as recited in claim 9 wherein said software employs frequency analysis.
